(19) United States
(12) Patent Application Publication
(10) Pub. No.: US 2011/0117190 A1
(43) Pub. Date: May 19, 2011

(54) Pharmaceutical Formulations

(75) Inventors: Adrian Brown, Essex (GB); Lee J. Gorringe, Hertfordshire (GB); Stephen Mark McAllister, Kent (GB); Wayne M. Matthews, Essex (GB); Danielle Genevieve Rebecca Russell, Essex (GB)

(73) Assignee: Glaxo Group Limited

(21) Appl. No.: 12/741,596

(22) PCT Filed: Nov. 7, 2008
(86) PCT No.: PCT/IB08/03872

§ 371 Date: Jan. 31, 2011

Related U.S. Application Data
(60) Provisional application No. 60/986,383, filed on Nov. 8, 2007.

(51) Int. Cl. A61K 9/48; A61K 9/00; A61K 47/38

(52) U.S. Cl. 424/452; 424/400; 514/781; 514/770; 514/772.5

(57) ABSTRACT

The present invention is directed to novel pharmaceutically acceptable polymeric compositions suitable for melt extrusion and injection moulding of single or multi-component pharmaceutical dosage forms comprising a plurality of drug substance containing sub-units, being capsule compartments and/or solid sub-units comprising a solid matrix of a polymer which contains a drug substance, the sub-units being connected together in the assembled dosage form.

### FIELD OF THE INVENTION

This invention relates to the preparation of injection molded single or multi-component dosage forms using novel pharmaceutically acceptable polymeric blends.

### BACKGROUND OF THE INVENTION

Various types of pharmaceutical dosage forms are known for oral dosing. Pharmaceutical capsules are well known, generally being intended for oral dosing. Such capsules generally comprise an envelope wall of a pharmaceutically acceptable, e.g. orally ingestible, polymer material such as gelatin, although other materials for capsule walls, e.g. starch and cellulose based polymers are also known. Such capsules generally have soft walls made by making a film on a capsule former, which is then allowed to dry. Rigid walled capsules made by injection molding are also known, see for example U.S. Pat. No. 4,576,284; U.S. Pat. No. 4,591,475; U.S. Pat. No. 4,655,840; U.S. Pat. No. 4,738,724; U.S. Pat. No. 4,738,817 and U.S. Pat. No. 4,790,881 (all to Warner Lambert). These disclose specific constructions of capsules made of gelatin, starch and other polymers, and methods of making them by injection molding of hydrophilic polymer—water mixtures. U.S. Pat. No. 4,576,284 specifically discloses such capsules provided with a cap which closes the capsule, and which is formed in situ on the filled capsule by molding. U.S. Pat. No. 4,738,724 discloses a wide range of rigid capsule shapes and parts.

Multi-compartment capsules, including those of the type where each compartment has different drug release characteristics, or for example, contains a different drug substance or formulation are also known, for example in U.S. Pat. No. 4,738,724 (Warner-Lambert); U.S. Pat. No. 5,672,359 (University of Kentucky); U.S. Pat. No. 5,443,461 (Alza Corp.); WO 95/16438 (Cortecs Ltd.); WO 90/12567 (Helminthology Inst.); DE-A-3727894, and BE 900950 (Warner Lambert); FR 2524311, and NL 7610038 (Tapanhony NV); FR 1,454,013 (Pluripharm); U.S. Pat. No. 3,228,789 (Glassman); and U.S. Pat. No. 3,186,910 (Glassman) among others. U.S. Pat. No. 4,738,817 discloses a multicompartment capsule with a similar construction to those of U.S. Pat. No. 3,228,789 and U.S. Pat. No. 3,186,910 made of a water-plasticized gelatin. U.S. Pat. No. 4,738,817 ('817) Witter et al., U.S. Pat. No. 4,790,881 ('881) Wittwer et al., and EP 0 092 908, Wittwer, F., all discloses injection molded capsules prepared with gelatin and other excipients. Wittwer et al. '817 and '881 also prepare capsules with other hydrophilic polymers, such as hydroxypropylmethyl-cellulose phthalate (HPMCP), methylcellulose, microcrystalline cellulose, polyethylene glycol, cellulose acetate phthalate (CAP) and with polyvinylpyrrolidone. Both U.S. Pat. No. 4,790,881 and EP 0 091 908 propose other polymers having enteric properties suitable for use, including generally acrylates and methacrylates (Eudragits) although none are demonstrated and no specific details are provided.

Pharmaceutical dosage forms are also known which comprise a matrix of a solid polymer in which a drug substance is dispersed, embedded or dissolved as a solid solution. Such matrixes may be formed by an injection molding process. This technology is discussed in Cuff G, and Raouf F, Pharmaceutical Technology, June (1998) pages 96-106. Some specific formulations for such dosage forms are disclosed in U.S. Pat. No. 4,678,516; U.S. Pat. No. 4,806,337; U.S. Pat. No. 4,764,378; U.S. Pat. No. 5,004,601; U.S. Pat. No. 5,135,752; U.S. Pat. No. 5,244,668; U.S. Pat. No. 5,139,790; U.S. Pat. No. 5,082,655; U.S. Pat. No. 5,552,159; U.S. Pat. No. 5,939,099; U.S. Pat. No. 5,741,519; U.S. Pat. No. 4,801,460; U.S. Pat. No. 6,063,821; WO 99/27909; CA 2,227,272; CA 2,188,185; CA 2,211,671; CA 2,311,308; CA 2,298,659; CA 2,264,287; CA 2,253,695; CA 2,253,700; and CA 2,257,547 among others.

U.S. Pat. No. 5,705,189 is directed to a group of co-polymers of methacrylic acid, methyl methacrylate and methyl acrylate, for use as thermoplastic agents in the production of drugs coatings, and capsules. No information is presented on the quality of the capsule formation with respect to warping or other distortions produced by the injection molding process. Nor is shear rate data presented for the viscosity/temperature figures of the emulsions presented therein.

It would be desirable to prepare a pharmaceutical dosage form in which a pharmaceutically acceptable polymeric blend is extruded by hot melt into a suitable dosage form, or is injection molded into suitable dosage forms which may be multicompartmental, such as in a capsule. This pharmaceutical polymeric composition as the dosage form may provide differing physio-chemical characteristics for each segment containing an active agent, such that a convenient dosage form can be optioned which may include a rapid dissolve, immediate, delayed, pulsatile or modified release, and be produced by simply selecting the appropriate polymer(s) to be molded for each section.

### BRIEF DESCRIPTION OF THE DRAWINGS

FIG. 1 demonstrates the dissolution profile for Metformin in a 60% HPMC-AS (LG)/20% Klucel EF/10% Triacetin/10% stearyl alcohol shell, using a USP III apparatus at 10DPM, 2 hours in pH 1.2 SGF, and 2 hours in pH 6.8 SIF.

FIG. 2 demonstrates the dissolution profile % Metformin Released (X-axis) of shells with HPMC-AS (LG)/Klucel EF/stearyl alcohol/Glycerol at 62.75/24.5/6/5/6/25% w/w, with a cellulose linker, using a USP III apparatus at 10DPM, 2 hours in pH 1.2 SGF.

FIG. 3 demonstrates a typical USP II release profile (dissolution profile) for paracetamol in 7.7×9.0 mm shells HPMC-AS/HPMC-P/HPC-SSL/Propylene Glycol/Glycerol/Stearyl Alcohol (58.5/18.5/3/10/5/5% w/w) with RL100 linkers, Run at 100 rpm in 0.1N HCl for 2 hours then pH 6.8.

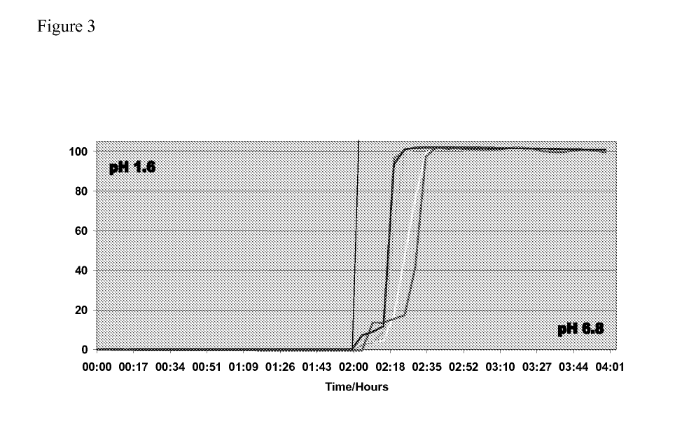

FIG. 4 demonstrates the dissolution profile for Metformin in a USP III Dissolution with shells of HPMC-AS/HP-50/SSL/Propylene Glycol/Glycerol/Stearyl Alcohol with an RL100 linker, run at 10 dpm in 0.1N HCl for 2 hours then pH 6.8 buffer.

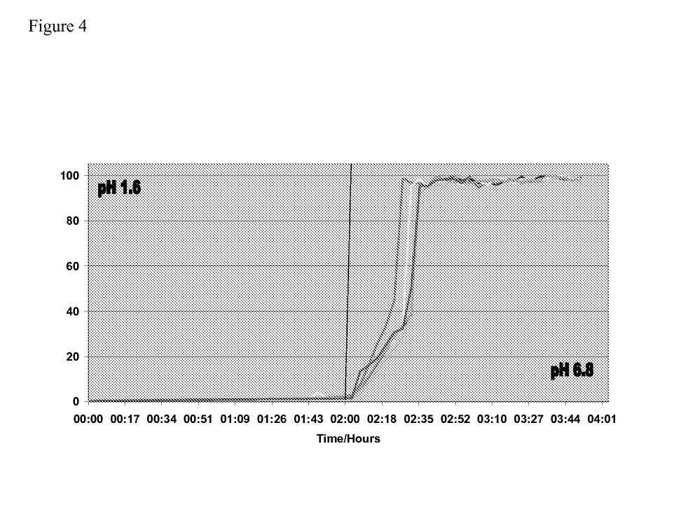

FIG. 5 demonstrates an extended USP III (6 hours acid) dissolution for Metformin with shell of HPMC-AS/HPMC-P (HP-50)/HPC-SSL/Propylene Glycol/Glycerol/Stearyl Alcohol, in a 0.4 mm wall thickness, 7.7×9.0 mm shells with RL100 linker, run at 10 dpm in 0.1N HCl for 6 hours (pH 1.6), then pH 6.8 phosphate buffer.

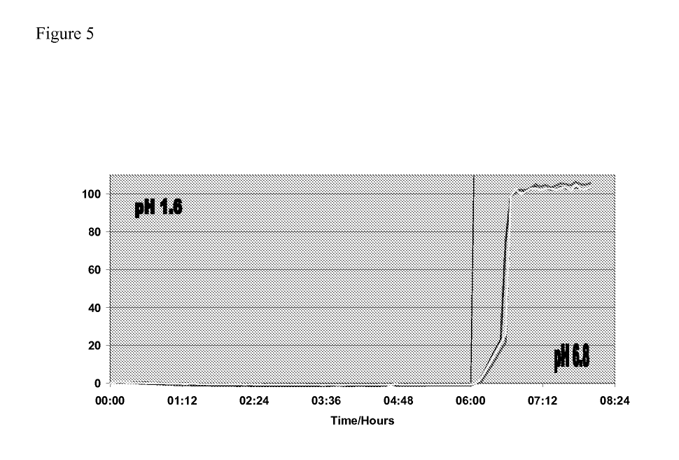

FIG. 6 demonstrates paracetamol release in a USP 2 Dissolution of Large Units 9×11 mm, 0.4 mm of an HPC-SSL Immediate release formulation (HPC-SSL/Opadry White/Glycerol/Stearyl alcohol/SDS 87/2/5/5/1% w/w) and an enteric shell (HPMC-AS/HP-50/SSL/Propylene Glycol/Glycerol/Stearyl Alcohol 58.5/18.5/3/10/5/5), at 100 rpm, at 2 hrs 10 mins in acid then at pH 6.8.

### SUMMARY OF THE INVENTION

The present invention is directed to a novel pharmaceutical composition for making moulded articles, such as capsule shells, solid sub-units, closures or linker sub-units comprising hydroxypropylmethylcellulose acetate succinate (HPMC-AS) present in an amount of about 20 to about 70% w/w; a plasticizer present in an amount of about 1% to about 20% w/w; a lubricant present in an amount of about 2% to about 10% w/w; at least one dissolution modifying excipient selected from a disintegrant, a swellable solid, or a wicking agent, or a combination or mixture thereof and wherein if the disintegrant is present it is in an amount of about 2% to about 20% w/w, and wherein if the swellable solid is present it is in an amount of about 10 to about 60% w/w, and wherein if a wicking agent is present it is in an amount of about 2.5 to about 15% w/w.

The present invention is also directed to the process of making the capsule shells, solid sub-units, closures or linker sub-units composed of the above formulation, and multi-component dosage forms composed of these assembled subunits, or other subunits of suitable formulations thereof.

### DETAILED DESCRIPTION OF THE INVENTION

In one embodiment the present invention is direct to a dosage form comprising at least one of:

(a) a shell including a first wall portion at least partially defining an interior space configured to hold a drug substance, the first wall portion being configured to dissolve within a gastrointestinal environment; or
(b) a linker including a second wall portion having a substantially cylindrical outer surface, the second wall portion configured to dissolve within a gastrointestinal environment;

wherein a respective one of the first or second wall portions are made from an extruded pharmaceutical composition comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

In another embodiment the present invention is directed to a capsule comprising a shell having an outer surface and an opposed inner surface, the inner surface defining at least in part a confined space for holding a drug substance, or a generally cylindrical linker body having an outer surface, the shell or the linker being composed of an extruded material comprising a pharmaceutical composition comprising hydroxypropylmethylcellulose acetate succinate (HPMC-AS) present in an amount of about 20 to about 70% w/w; at least one plasticizer present in an amount of about 1% to about 15% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

Another embodiment of the invention is a dosage form component configured as a hollow capsule, an end cap, or a linker, said component consisting essentially of an extruded or injection moulded pharmaceutical composition comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

Another embodiment of the invention is a dosage form, comprising:

a) a capsule shell including a wall at least partially defining an interior space for retaining a drug substance and being configured to dissolve within a gastrointestinal environment; and

b) a linker including a wall having a substantially cylindrical outer surface and being configured to dissolve within a gastrointestinal environment;

wherein at least one of the capsule shell or the linker is made from an extruded material comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

Therefore, one embodiment of the invention is a dosage form comprising at least one of:

(a) a shell including a first wall portion at least partially defining an interior space configured to hold a drug substance, the first wall portion being configured to dissolve within a gastrointestinal environment; or
(b) a linker including a second wall portion having a substantially cylindrical outer surface, the second wall portion configured to dissolve within a gastrointestinal environment;

wherein a respective one of the first or second wall portions are made from an extruded material comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

Another embodiment of the invention is a dosage form apparatus comprising a wall portion configured to be dissolvable within a gastrointestinal environment, the wall portion made from an extruded material comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

Another embodiment of the invention is a dosage form comprising at least one subcomponent having a wall portion made from an extruded material comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

The present invention provides for novel pharmaceutical compositions, and their use in melt extrusion technologies, and in the making of injection molded articles, such as capsule shells, linkers, spacers, and multicomponent injection molded capsule shells, linkers or spacers, multicomponent pharmaceutical dosage forms, and other aspects as defined in the claims and description of this application.

Another embodiment of the invention is to provide an alternative and improved pharmaceutical dosage form which provides, inter alia, greater flexibility in the dosage form adapted to a patient's specific administration requirement, using the novel formulations of pharmaceutically acceptable polymers and suitable excipients in said dosage forms.

Another embodiment of the invention is to provide a process of producing the multicomponent dosage forms comprising the novel pharmaceutically acceptable polymeric blends by injection molding. These multi-component dosage forms are suitable for containing a pharmaceutically acceptable active agent, or agents, for release thereby.

In accordance with the invention, a melt extrusion composition, and an injection molded capsule shell, and/or linker is provided for, with a composition of hydroxypropyl methylcellulose acetate succinate (HPMC-AS) and additional excipients.

In one embodiment of the invention, the capsule or linker subunits comprises hydroxypropyl methylcellulose acetate succinate present in an amount of about 10 to about 80% w/w, in combination with various other excipients to product a formulation that can be first extruded, and if desired injection moulded. The composition further comprises a dissolution-modifying excipient (DME) present in an amount of 2.5% w/w to about 60% w/w as determined by classification of DME; and a lubricant present in an amount of about 1 to about 10% w/w, suitably from about 2 to about 10 w/w; and optionally a plasticizer present in an amount from about 1% to about 15% w/w, and optionally a processing agent present in an amount from about 1% to about 10% w/w.

In an alternative embodiment, the HPMC-AS is present in an amount of about 20 to 70% w/w, alternatively from about 40 to about 70% w/w, and alternatively in an amount of about 55 to about 65% w/w, and alternatively in an amount about 60% w/w.

One embodiment of the present invention is the use of these injection moulded parts which are resistant to gastric fluids, but deform and dissolve in the higher pH of intestinal fluid and therefore provide a mechanism for release of the contents of the these injection moulded, orally dosed, capsules in the intestine.

In an alternative embodiment, the pharmaceutical dosage form comprises a plurality of sub-units, each being a drug substance-containing capsule compartment. In this case, each compartment is physically separated from at least one adjacent compartment, preferably by a wall made of a pharmaceutically acceptable polymer material. In the case in which at least one of the sub-units is a drug substance-containing capsule compartment its wall thickness is in the range of about 0.1-0.8 mm. In another embodiment the wall thickness is in the range of about 0.3-0.8 mm. In another embodiment the wall thickness is in the range of about 0.3-0.5 mm.

The multi-component dosage form of the invention affords a high degree of versatility in that it can be composed of various combinations of different dosage forms having different release characteristics. For example, the sub-units can be a substantially immediate release sub-unit, a sustained release sub-unit, or a pulsed release sub-unit.

Other objects and advantages of the invention will be apparent from the following description.

The present invention is directed to novel compositions of a pharmaceutically acceptable polymer, hydroxypropyl methylcellulose acetate succinate (HPMC AS) and pharmaceutically acceptable excipients, which polymeric composition may be injection molded into one or more components which can optionally be utilized together, such as in a stacked or multi-component dosage form. It is recognized that the polymeric blends may be injection molded into a single component that may also contain the active agent for oral administration in the moulded component, or the moulded component(s) may contain the active agent within its cavities.

The present invention also relates to the application of a pharmaceutically acceptable film coating over a component comprising the novel pharmaceutically acceptable polymeric blends as described herein. The film coating may be a delayed release coating, or a pH control coating as are well known in the art. Such suitable coatings include but are not limited to HPMC coatings, such as Opadry, and Eudragit coatings, such as L30D-55. Enteric coatings, represented by application of L30D-55 for instance, may be applied using standard equipment such as a GMP Aerocoater column coater. The component weight gain is nominally from about 3% to about 5% w/w.

A desired attribute of the pharmaceutically acceptable polymeric blends herein is to provide a consistent dissolution profile in vitro and optimally in vivo.

A suitable multicomponent dosage form is disclosed in WO 01/08666, and other related applications on structural features, or associated film coatings, etc. for use with components or subunits of the above noted formulations may be found in WO 01/08666; WO 04/010978, PCT/EP08/63852 (Attorney Docket No. PU62554), PCT/EP08/63853 (Attorney Docket No. PU62555), PCT/EP08/63856 (Attorney Docket No. PU62556), and PCT/EP08/63857 (Attorney Docket No. PU62557) all filed 15 Oct. 2008.

Suitable formulations which may be used to derive parts of a dosage form which may be used with part of a dosage form of this invention, e.g. a capsule compartment wall, a solid sub-unit, or a closure or linker sub-unit are disclosed in WO 02/060385, WO 02/060384, WO 05/089726, WO 05/009380, and U.S. Ser. No. 61/061,275 filed 13 Jun. 2008 (Attorney Docket No. PU62992P).

The parts of the dosage form of this invention, e.g. a capsule compartment wall, a solid sub-unit, or a closure or linker sub-unit, comprise a pharmaceutically acceptable polymeric blend (and adhesive material if adhesive welds are formed) which is generally regarded as safe, e.g. for oral ingestion and is capable of being formed into the required shape of a capsule compartment wall, a solid sub-unit, or a closure or linker as described above. A preferred method of forming the polymer material into the desired shape is injection molding, which may be a hot or cold runner injection molding process. Suitable injection molding machines for such a process are known.

The pharmaceutical dosage form may comprise a plurality of capsule compartments each bounded and physically separated from at least one adjacent compartment by a wall made of a pharmaceutically acceptable polymer material, such as described herein, adjacent compartments being connected together in the assembled dosage form, and being retained together by the connection at least prior to administration to a patient, one or more of the compartments containing a drug substance. Suitably, in the assembled dosage form of this first embodiment there are at least two, for example three, such capsule compartments. Three or more such compartments may be linearly disposed in the assembled dosage form, e.g. in an arrangement comprising two end compartments at opposite ends of the line, and one or more intermediate compartments. Suitably, there may be two such capsule compartments. Suitably, one of such two capsule compartments may be made of a material which is a sustained release component, i.e. so that the capsule compartment wall dissolves, bursts or is otherwise breached to release its contents after a delay, e.g. when the compartment has reached the intestine. Suitably, the other of such two capsule compartments may be made of a material which is an immediate release component, i.e. so that the capsule compartment wall dissolves, bursts or is otherwise breached to release its contents immediately or effectively immediately, e.g. when the compartment is in the mouth or stomach.

One or more, e.g. all, of the capsule compartments may for example be substantially cylindrical, which term includes shapes which have a circular, oval or oblate circular cross section across the longitudinal axis, and shapes which have parallel or tapering e.g. with side walls which taper conically over at least part of their extent. Such substantially cylindrical capsule compartments may be provided with connectable parts at one or both of their longitudinally disposed ends so that the assembled dosage form may also be overall of a substantially cylindrical shape.

Various blends of polymers, such as the methacrylic acid copolymers (i.e., Eudragit E®, Eudragit E100® Eudragit® L and/or Eudragit® S), poly(meth)acrylate copolymers (such as Eudragit® 4135F, and 4155F) and ammonium methacrylate copolymers (such as Eudragit® RL and/or Eudragit® RS) have been used for hot melt extrusion and injection molding.

Acrylic and/or methacrylic acid-based polymers which are soluble in intestinal fluids and which can be formed into capsules are for example disclosed in U.S. Pat. No. 5,705,189 (Roehm GmbH) which is incorporated herein by reference. These poly(meth)acrylate copolymers were extrudable and injection molded into capsule half's wherein the ratio of acrylic and/or methacrylic acid was generally 20% w/w or more of the copolymer (Examples 1-8). In these Examples, glycerol monostearate was added on a 16% w/w basis of the polymer as the sole mold-releasing agent.

In one embodiment of the invention herein, in order to produce injection molded, non-distorted, unwarped capsule/sub-unit components for assembly into either single capsule or multicompartment dosage forms using HPMC-AS, at least one lubricant and a dissolution modifying agent are included in the formulation as being useful to obtain release from the injection molds.

HPMC-AS is the base polymer in the formulations described herein that provides an enteric-like functionality to the injection moulded parts. HPMC-AS is available in granular and in a fine micronised form in three grades from Shin-Etsu Chemical Co. Ltd as Aquoat® AS-LG/LF, Aquoat® AS-MG/MF and Aquoat® AS-HG/HF. The different grades are defined by the number acetyl and succinoyl groups that are introduced to the hydroxyl groups on the backbone of the polymer. L grades have an acetyl content of 5.0%-9.0% and a succinoyl content of 14.0%-18.0%. M grades have an acetyl content of 7.0%-11.0% and a succinoyl content of 10.0%-14.0%. H grades have an acetyl content of 10.0%-14.0% and a succinoyl content of 4.0%-8.0%. All three of these grades are demonstrated in the working examples herein.

It is recognized that HPMC-AS may be blended with other pharmaceutically acceptable polymers, such as those described in detail in the Handbook of Pharmaceutical excipients, published jointly by the American Pharmaceutical association and the Pharmaceutical society of Britain.

A number of different excipients were evaluated for use in with HPMC-AS to create an enteric shell having favourable dissolution profiles, physical stability, chemical stability, tensile strength and ease and reproducibility of manufacture.

The HPMC-AS polymer is blended with additional excipients which include, but are not limited to, lubricants, such as stearyl alcohol; swelling agents, such as hydroxypropylcellulose, etc.; surfactants, such as SDS or the Pluronic group of agents; pore-forming/channeling agents, such as lactose or PEG; and additional buffering agents for adjust of microclimate pH conditions.

Dissolution modifying agents, or substances are those that assist in release modification, alter the erosion and/or swelling characteristics of the capsule shell/linker/component. Many different classes of agents may be used, such as the known super-disintegrants represented by sodium starch glycollate, Ph. Eur. or sodium carboxymethyl starch, JPE (“Explotab”®, produced by JRS Products), croscarmellose sodium NF (Aci-Di-Sol® produced by FMC), cross-linked PVP (“Kollidon-CL”), and copovidone (“Kollidon VA 64”), both commercially available from BASF, Starch 1500, and swelling agents such as polyvinyl pyrrolidone (PVP, also know as POVIDONE, USP), manufactured by ISP-Plasdone or BASF-Kollidon, primarily Grades with lower K values (K-15, K-25, but also K-30 to K-90); and crospovidone (cross-linked polyvinyl pyrrolidone); and combinations or mixtures thereof. Kollidan VA 64, or copovidone, is also known as copolyvidone, copovidonum, copovidone or copovidon and is a ratio of two monomers, vinylpyrrolidone and vinyl acetate.

Suitably, this class of disintegrants are present in the range of about 2 to 20%, alternatively from about 5 to 10% w/w.

Another class of agents of dissolution modification agents for use herein are the swellable solids, and include but are not limited to poly(ethylene)oxide; the cellulosic derivatives, such as cellulose acetate phthalate; hydroxypropylcellulose (HPC), such as the lower molecular weights, e.g., KLUCEL EF and LF grades, and mixtures of the lower molecular weights with higher molecular weight grades such as JF or GF or alternative suppliers of HPC such as Nippon Soda Company, or Nisso HPC, having a grade HPC-SSL; hydroxypropylmethyl cellulose (HPMC), and hydroxypropylmethylcellulose phthalate (HPMCP), and other hydroxyalkylcellulose derivatives. At least one commercial source of hydroxypropylmethylcellulose phthalate is available from Shinetsu, Japan.

One source of HPC is marketed by Aqualon, a division of Hercules Incorporated, as Klucel®. Klucel HPC is produced in various grades, as determined by their intended use. Suitable Klucel polymers are Klucel EF, Klucel JH, Klucel LF, and Klucel GF. Klucel E has a viscosity in the range of 150-700 (a 300-600 mPas for EF pharm/EXF Pharm), and a molecular weight of about 80,000; J has a viscosity of 150-400 and a molecular weight of about 140,000, L has a viscosity in the range of 75-150, and a molecular weight of about 95,000; and G has a viscosity in the range of 75-400, and a molecular weight of about 370,000.

One commercially available HPMC is Pharmacoat™ 603. Pharmacoat™ is Hypromellose USP, produced by Shines, Chemical Company. Hypromellose is also referred to as hydroxypropylmethylcellulose, and for purposes herein used interchangeably. Pharmacoat 603 has a substitution type of 2910 USP designation, and a labelled viscosity (cP or mPa's) of 2.4 to 3.6, a moisture permeability of 207, a methoxyl content of 28.0 to 30.0%, and a hydroxypropoxyl content of 7.0-12.0% (USP). An alternative source of commercially available hypromellose having similar viscosity, and substitution is Opadry™ from Colorcon, N.J., USA, or Methocels from Dow Chemical Company, Midland, Mich.

Suitably, these swellable solids are present in the range of about 10% to about 60% w/w. In another embodiment the swelling agent is present in an amount from about 20 to about 30% w/w, or alternatively from about 10 to about 50% w/w. It is recognized that more than one swellable solid may be used in combination in the formulations of this invention.

Therefore, one embodiment of the invention is a co-blend of HPMC-AS with the polymer hydroxypropyl cellulose (HPC). In one embodiment of the invention the coblend of HPMC-AS is with a swellable solid that is a blend of at least two hydroxypropylcellulose derivatives each having differing molecular weights.

One embodiment of the invention is a co-blend of HPMC-AS with the polymer HPC having a viscosity in the range of 150-700, such as Klucel EF. Suitably when Klucel EF is used as a DME it is in the range of 10 to 47.5% w/w.

Addition of these thermoplastic polymers to the blend is believed to provide for improved tensile properties over HPMC-AS alone both pre and post hydration, and enables swelling of the polymer at a pH of 1 to 6.

The co-blended polymers of HPC and HPMC-AS produce shells which hydrate more than the non-blended polymeric composition (HPMC-AS alone) under gastric conditions. This produces a formulation which has significant improvements in dissolution reproducibility; an enhanced hydration profile which results in less structural integrity, in alkaline media, upon dissolution; and appearance and tensile properties of the resulting shells.

Another embodiment of the invention is a co-blend of HPMC-AS with the swellable solid hypromellose phthalate (HPMC-P or HPMCP) such as that marketed by Shin Estu, as HP-50, HP-55, HP-55S®. Hypromellose phthalate NF is also referred to as hydroxypropylmethylcellulose Phthalate JP and is used interchangeably herewith. The viscosity of the HP-55 is 40 cSt, with a Nominal Phthalyl content 31%, a mean particle size (um) of 1000 and dissolves in pH>or=to 5.5. HP55S is similar but for a viscosity of 170 cST. The HP-50 is 55 cSt, with a Nominal Phthalyl content 24%, a mean particle size (um) of 1000 and dissolves in pH>or=to 5.0.

Suitably if HPMCP is present in it is in the range of 10 to about 50% w/w, suitably from 15 to 30%, and in another embodiment it is present in an amount of about 20 to 25% w/w. In one embodiment the HPMCP is HP50.

HP-50 has the lowest molecular weight and hence the lowest viscosity. This was demonstrated to make processing easier, and HP-50 also contains the least amount of phthalic acid groups, perhaps providing lower long term chemical instability.

HP-55 also dissolves at the higher pH (5.5 versus 5.0 for HP-50) which could result in a longer release time in vivo if the pH rise is not sufficient. HP-55S is the highest viscosity grade of HP-55, and hence causes a bigger increase in torque and pressure on manufacture, which may lead shells having higher levels of degradation. In general, shells containing HP-50 appear to be more stable than either HP-55 or 55S and dissolve faster.

In another embodiment of the invention the combination is suitably HPMC-AS LG in combination with HPMC-phthalate (HPMCP) for formulation of the capsule shell wall. In another embodiment the HPMC-AS is present in an amount of about 50 to about 65% w/w, and an optimal ratio of HPMC-AS:HPMCP is around 3:1.

Suitably, one formulation of the invention is HPMC-AS present in an amount of about 50 to about 65% w/w, HPMCP is present in an amount from about 15 to about 30% w/w. In another embodiment, HPMC-AS present in an amount of about 50 to about 65% w/w, HPMCP is present in an amount from about 15 to about 30% w/w, stearyl alcohol present in an amount of about 4 to about 10% w/w, and at least one plasticizer is present in an amount of about 10 to about 20% w/w. In one embodiment the plasticizer is selected from glycerol or propylene glycol, or a mixture thereof. In another embodiment the plasticizer is selected from TEC or propylene glycol, or a mixture thereof.

Another embodiment of the invention is a co-blend of HPMC-AS, HPC and a second swellable solid, such as HPMC. HPMC suitably is present in this co-blend in an amount of about 2 to about 10% w/w.

In another embodiment of the invention there is a co-blend of HPMC-AS, HMPCP, and a second swellable solid, HPMC. The HPMCP suitably is present in this co-blend in an amount of about 15 to about 30% w/w. HPMC suitably is present in this co-blend in an amount of about 2 to about 10% w/w.

In another embodiment of the invention there is a co-blend of HPMC-AS, and HPC, suitably HPC-SSL. The amount of HPC-SSL in the blend is present from about 3 to about 25% w/w.

In another embodiment of the invention there is a co-blend of HPMC-AS, and HPC-SSL, and a second swellable solid, HPMCP. HPMCP suitably is present in this co-blend in an amount of about 15 to about 30% w/w, and the amount of HPC-SSL in the blend is from about 3 to about 20% w/w.

In another embodiment of the invention there is a co-blend of HPMC-AS, and HPC-SSL, a second swellable solid, HPMCP, and a third swellable solid HPMC, such as Pharmacoat 603. In this blend, the HPMC-AS is present from about 45 to about 60% w/w; the HPMCP is present in this co-blend in an amount of about 15 to about 20% w/w, the amount of HPC-SSL in present in the blend from about 1 to about 20% w/w, suitably about 3% w/w to less than 20% w/w, and alternatively form about 1 to about 5% w/w; and the HPMC is present in the blend from about 3 to about 5% w/w.

Hydroxypropyl cellulose is suitably added to the blend to help processing and injection moulding of the shells, to give better tensile properties, and assist in dissolution of the shell matrix in a pH independent manner.

Addition of an HPC such as Klucel EF, has been shown to result in moulded shells but which have longer dissolution times in high pH media due to the swelling nature and relatively low solubility rate of Klucel, e.g. swelling versus erosion.

Addition of a lower molecular weight HPC, such as HPC-SSL has been shown to increase dissolution rates at higher pH, and increases flexibility of shells to enable clipping after storage. If the level of HPC-SSL is increased too much, the polymer matrix became too soluble in acidic pH and the shells may fail in enteric testing, therefore, suitably inclusion of HPC-SSL in present in the formulations in amounts of 1% to about 25%, suitably, less than 20%.

Additionally, the presence of small amounts of HPC-SSL (1-5% w/w) has also found to help stabilise HPMC-P in the preferred formulation compared to formulations without this addition.

Addition of HPMC, such as Pharmacoat 603 appears to help improve extrusion of the formulations. However, certain components containing Pharmacoat 603 have been shown over time to become brittle. It has been found that using HPMC in place of HPC-SSL in the formulations slows down the dissolution rate. Samples with addition of 3% w/w SSL release between 24-36 minutes at high pH, and this is increased to 36-72 minutes when the SSL is replaced with 5% w/w HPMC.

Shells comprising of all HPMC-AS as the enteric polymer with complete removal of the phthalate, to help improve stability have been tested under USP 3 conditions. The dissolution times have been shown to be more variable and tend to be longer, although the shells behaved very similar under USP 2 conditions. Consequently, while the all HPMC-AS shells can be extruded and moulded, it is preferred that a co-blended of polymers be used for enteric shells having better tensile properties.

Other suitable dissolution modifying excipients include, but are not limited to the class of wicking agents such as the low molecular weight solutes, such as starch, or the non-reducing sugars, such as xylitol, or mannitol, present in the range of about 2.5 to about 15% w/w. Also included herein are the class of water soluble fillers, such as lactose, lactitol, maltitol, sorbitol or alternatively organic acids such as malic acid, citric acid or succinic acid, suitably present in the range of about 2.5 to about 15% w/w, alternatively from about 5 to about 10% w/w. In another embodiment of the present invention the water soluble fillers may be present from an amount of about 5 to about 20% w/w.

It is recognized that the polymeric compositions are first melted in a melt extrusion process, and may also contain additional additives or excipients to assist in melt flow, strength, brittleness, flexibility, elasticity, and other moulding characteristics, these additional excipients include but are not limited to, plasticizers, absorption enhancers, surfactants, flavouring agents, dyes, absorption enhancers, lubricants, additional dissolution modifying agents, processing aids, colouring agents, flavouring agents and sweetening agents, etc.

Incorporation of a surfactant into the formulation may optionally be desired to lower the viscosity and surface tension of the formulation. The surfactant selection may be guided by HLB values but is not necessarily a useful criterion. Higher HLB surfactants are Tween® 80 (HLB=10), Pluronic F68 (HLB=28), and SDS (HLB>40); lower HLB value surfactants, such as Pluronic F92 and F127 may also be used. Pluronic, made by BASF, USA has a synonym of POLOXAMER. Pluronic F68 for instance has a molecular weight of 8,400. Pluronic F127 has a molecular weight of 12,600. Pluronics are polyoxypropylene-polyoxyethylene block copolymers.

A surfactant may also be called an oligomeric surface modifier and includes, but is not limited to: Pluronics® (block copolymers of ethylene oxide and propylene oxide, and are also referred to as polyoxypropylene-polyoxyethylene block copolymers); lecithin, Aerosol OT® (sodium dioctyl sulfosuccinate), sodium lauryl sulfate, Polyoxyl 40® hydrogenated castor oil, polyoxyethylene sorbitan fatty acid esters, i.e., the polysorbates such as Tween®, such as Tween 20, 60 & 80, the sorbitan fatty acid esters, i.e., sorbitan monolaurate, monooleate, monopalmitate, monostearate, etc. such as Span® or Arlacel®, Emsorb®, Capmul®, or Sorbester®, Triton X-200, polyethylene glycol's, glyceryl monostearate, Vitamin E-TPGS® (d-alpha-tocopheryl polyethylene glycol 1000 succinate), sucrose fatty acid esters, such as sucrose stearate, sucrose oleate, sucrose palmitate, sucrose laurate, and sucrose acetate butyrate, etc.; and combinations and mixtures thereof. Sodium lauryl sulfate, may also be referred to herein as sodium dodecyl sulfate (SDS).

Suitably, the formulation may optionally contain from about 1% to about 10% w/w surfactant(s). In another embodiment the formulation contains from about 1 to about 8% w/w surfactant(s). If SDS is added it is suitably around 1% w/w. If Tween 80 is added it is around 2% w/w or less, alternatively from about 0.5 to about 2% w/w.

The polymeric carriers or oligomeric surface modifiers, if appropriately chosen, may themselves act as absorption enhancers. Suitable absorption enhancers for use herein, include but are not limited to, chitosan, lecithin, lectins, and Vitamin E-TPGS, and combinations or mixtures thereof. Suitably, these absorption enhancers if present are in a range of about 1 to about 20% w/w.

Plasticizers may be employed to assist in the melting characteristics of the composition. A plasticizer may increase the flexibility of the moulded parts and reduces the melt viscosity which then aids the extrusion and injection moulding process. Various plasticizers were found to plasticize the enteric polymers (HPMC-AS and HPMC-P) to varying degrees, each plasticizer had its own benefits and drawbacks on the desired critical attributes for the enteric dosage form.

Suitable plasticizers that may be employed in this invention are triethyl citrate (TEC), triacetin, tributyl citrate, acetyl triethyl citrate (ATEC), acetyl tributyl citrate (ATBC), dibutyl phthalate, dibutyl sebacate (DBS), diethyl phthalate, glycerol, vinyl pyrrolidone glycol triacetate, polyethylene glycol, polyoxyethylene sorbitan monolaurate, propylene glycol, fractionated coconut oil, or castor oil; and combinations or mixtures thereof.

Triethyl citrate is a good plasticizer of HPMC-AS and HPMC-P providing shells with good physical properties, and an adequate dissolution profile with few enteric failures, and a high pH release occurring typically within 45 minutes.

It has been found that for formulations containing HPMCP, the plasticizer TEC has shown some chemical instability, likely due to the acidic nature of the TEC. It is believed that as HPMC-Phthalate degrades, the phthalic acid functional group is removed from the cellulose backbone, and this can alter the chemistry of the polymer and change its pH response. Suitably, the presence of phthalic acid in the formulations is limited to less than 1% of the HPMC-P polymer present for long term stability.

In one embodiment of the present invention the plasticizer triacetin is used in combination with the HPMC-AS co-polymer blend.

In another embodiment of the present invention the plasticizer triethyl citrate is used in combination with the HPMC-AS co-polymer blend.

In another embodiment of the present invention the plasticizer glycerol is used in combination with the HPMC-AS co-polymer blend. In another embodiment of the invention the plasticizer glycerol is used for formulations which further comprise as a copolymer blend with HPMC-AS, an HPMC-P component (HP-50).

In another embodiment of the present invention the plasticizer propylene glycol is used in combination with the HPMC-AS co-polymer blend.

Suitably, the plasticizer is present in an amount of about 1 to about 20% w/w, suitably about 1 to about 15% w/w. In one embodiment of the invention the plasticizers are present in an amount from about 2.5 to about 15% w/w, in combination of mixtures thereof. In another embodiment the plasticizer is present in an amount from about 5 to about 10% w/w.

If a single plasticizer is used, such as triacetin, suitably it will be in an amount of about 2.5 to about 15% w/w, and 4 to 10%; and 5 to 8%. If triethyl citrate is use, suitably it is in the range 2.5 to about 15% w/w, and 4 to 10%; and 5 to 8%.

If the plasticizer is glycerol, suitably it will be in an amount of about 2.5 to about 15% w/w, 5 to 13%; and 5 to 8%.

If the plasticizer is propylene glycol, suitably it will be in an amount of about 4 to about 15% w/w, and from 4 to 10% w/w.

In another embodiment of the invention a combination of plasticizers are used, such as propylene glycol with TEC or glycerol with propylene glycol. The amount of plasticizers in combination may be slightly higher than the individual component, suitably from about 1 to about 20% w/w. In another embodiment from about 10 to about 20% w/w, more suitably together about 15% w/w.

Replacement of propylene glycol and glycerol with polyethylene glycol (PEG) 400 provides shells with a reasonably reproducible dissolution profile, and a high level of enteric protection. However, generally the moulded shells showed poorer tensile properties, and difficulty with clipping to linkers. This could mean that PEG 400 is simply not an effective plasticizer for one or more of the enteric polymers present in a co-blended formulation.

It has been determined that when the polymer HPMC-AS LG is used in combination with the plasticizer triacetin that an optimal ratio of HPMC:triacetin is from about 4:1 to about 7:1, preferably closer to 7:1. In such cases the lubricant, preferably stearyl alcohol, is suitably maintained at approximately 5-7% w/w to the total formulation with the remainder of the formulation being the dissolution modifying excipient/agent, and any other additives as necessary. Suitably, the DME is a swellable solid, preferably HPC or blends of HPC. In one embodiment the HPC polymer is Klucel EF.

The use of triacetin has demonstrated good extrusion, moulding and overall effective plasticization with the enteric cellulosic polymers herein. Shells with higher levels of triacetin are shown to be more unstable on store but not an issue at the levels commonly used herein.

Additional regents, generally classified as processing aids, include strengthening agents, such as talc. Suitably, the processing aids are present from about 0.5 to about 10% w/w. In another embodiment, the processing aids are present from about 0.5 to about 5% w/w.

An internal lubricant is one which can provide lubrication at the die wall in the extrusion process, and mould wall in the injection moulding process. Suitable mould processing lubricants, or glidants for use herein, include but are not limited to, stearyl alcohol, stearic acid, glycerol monostearate (GMS), talc, magnesium stearate, silicon dioxide, amorphous silicic acid, and fumed silica; lauric acid, lecithin, sucrose fatty acid esters such as those derived from stearic acid, oleic acid, palmitic acid, and lauric acid; and combinations or mixtures thereof. It is believed that the lubricant functions primarily as a flow promoter for the composition. One embodiment of the present invention is the use of stearyl alcohol as a suitable lubricant. Suitably, a commercial grade of stearyl alcohol, such as Crodacol S95 (Croda Oleochemicals) is used herein. Suitably, a commercial grade of sucrose fatty acid esters such as those derived from stearic acid, oleic acid, palmitic acid, and lauric acid may be obtained from Mitsubihi-Kasei Foods as SurfHope®. The amount of lubricant present in the formulation is from about 2% to about 10% w/w. In another embodiment the lubricant is present from about 4% to about 8% w/w.

If stearyl alcohol is used, it is suitably present in an amount of about 2.0 to 10% w/w. In another embodiment the stearyl alcohol is suitably from about 4 to about 8% w/w. In another embodiment the stearyl alcohol is suitably from about 5 to about 7% w/w. In another embodiment the stearyl alcohol is suitably about 5 to about 6.25% w/w.

Suitably, the lubricant should act as a mould processing lubricant and cause little mold distortion, i.e. crumpling of the multidosage compartment shell when the hot soft shell is taken out of the mould. Suitably, the lubricants for use herein do not introduce any metal ion contamination.

One embodiment of the present invention is the combination of the polymer HPMC-AS, stearyl alcohol, at least one swellable solid, and at least one plasticizer. The swellable solid may be the polymer hydroxypropylcellulose or a blend of hydroxypropylcellulose derivatives; or the swellable solid may be HPMCP; or the swellable solid my be HPC-SSL; or the swellable solid may be HPMCP and HPC; or the swellable solid may be a blend of HPMCP and HPC-SLL; or the swellable solid may be a blend of HPMCP, HPC-SLL and HPMC.

In one embodiment an optimal ratio of HPMC-AS: HPC is in the range of 0.8:1. Levels of 0.5:1 may produce suitable part with reduced release time and reliable enteric performance.

Another embodiment of the present invention is the combination of the polymer HPMC-AS, stearyl alcohol, at least one swellable solid, and at least two plasticizers. The combination of plasticizers are suitably propylene glycol with TEC, or glycerol with propylene glycol.

While the compositions herein may be moulded in varying wall-thickness, it is preferably that capsules or components have a wall-thickness of about 0.3 to about 0.8 mm, suitably 0.4-0.5 mm. However, dissolution performance will more appropriately tailor the wall thickness depending upon the release profiles desired. Increases in wall thickness may be necessary to reduce warping of the components, or modification of the additional excipients in addition to this may be necessary.

The final products of this invention, i.e. the capsule shells, and or other components and sub-units may additionally include materials in the polymer blends of which they are made to enhance the ease with which they can be welded together. The sub-units may additionally be provided with constructional features and/or include materials in the polymer materials of which they are made to enhance the ease with which they can be joined together, either by simple mechanical joints, or welded together. A suitable material for assisting such are opacifier materials such as carbon (e.g. 0.2-0.5%), iron oxides, Ferric oxide (e.g. 0.2-0.5%), or titanium dioxide (e.g. 0.5-1.0%) which help the polymer.

For example each of a plurality of sub units, e.g. of the capsule compartments, linker sub-units, or combinations thereof may comprise the same or different polymer(s). For example each of a plurality of sub units, e.g. of capsule compartments, linker sub-units, or combinations thereof may comprise the same or different drug substance. For example each sub-unit may contain the same drug substance but release the contents into the gastro-intestinal tract of the patient at a different rate, at different times after administration to the patient or at different places in the patient's gastrointestinal system. Alternatively each sub-unit may contain a different drug substance, each of which may be released at the same or a different rate or time after administration or place in the patient's gastro-intestinal system.

For example two or more sub-units, e.g. two capsule compartments or a linker may each contain different drug substances, and/or different drug substance formulations, and/or the same drug in different formulations, so that a combination of two or more drug substances having different release rate profiles, or formulations thereof, may be administered to a patient.

The dosage form of this invention enables the assembly together of sub-units which differ in their drug content and/or drug content release characteristics to provide a dosage form tailored to specific administration requirements.

The dimensions and shape of each of the sub-units and hence of the overall assembled dosage form may be determined by the nature and quantity of the material to be contained therein and the intended mode of administration and intended recipients. For example a dosage form intended for oral administration may be of a shape and size similar to that of known capsules intended for oral administration.

The dosage form is particularly suitable for presentation as an oral dosage form containing one or more drug substances suitable for oral administration, and appears to be suitable for all types of such drug substance.

The drug substance(s) contained in any capsule compartment may be present in any suitable form, e.g. as a powder, granules, compact, microcapsules, gel, syrup or liquid provided that the capsule compartment wall material is sufficiently inert to the liquid content of the latter three forms. The contents of the compartments, e.g. drug substances, may be introduced into the compartments by standard methods such as those used conventionally for filling capsules, such as dosating pins or die filling.

The sub-units may differ from each other in their drug content release characteristics, and this may be achieved in various ways. For example, one or more solid sub-units and/or capsule compartments may be substantially immediate release, i.e. releasing their drug contents substantially immediately upon ingestion or on reaching the stomach. This may for example be achieved by means of the matrix polymer or the capsule compartment wall dissolving, disintegrating or otherwise being breached to release the drug content substantially immediately. Generally, immediate-release sub-units are preferably provided by being capsule compartments. The other subunit may alternatively be an immediate release subunit which comprises an enteric coating over the subunit.

For example, one or more solid sub-units and/or capsule compartments may be sustained-release sub-units. Preferably these are solid sub-units, as a bulk matrix of polymer is likely to dissolve or disperse more slowly to release its drug content that a thin walled capsule.

For example, one or more solid sub-units and/or capsule compartments may be pulsed-release sub-units for example releasing their drug content at a specific predetermined point in a patient's gastro-intestinal system. This may be achieved by the use of polymer materials which dissolve or disperse only at defined pH environments, such as the above mentioned HPMC-AS or certain Eudragit® polymers, for instance Eudragit E100 which is acid labile.

For example in the above-described capsule compartment-linker-capsule compartment dosage form one capsule compartment may be effectively immediate release and the other may be sustained, delayed or pulsed release. To achieve this for example one capsule compartment may be made of polymer materials which cause the capsule compartment to release its drug content in the stomach or upper part of the digestive tract, and the linker (acting as a closure for the second compartment) and the second compartment itself may be made of materials e.g. the above described enteric polymers, which release their drug content only in the intestinal environment.

Determination of the time or location within the gastro-intestinal tract at which a sub-unit releases its drug substance content may be achieved by for example the nature of the sub-unit material, e.g. a solid sub-unit matrix polymer or a capsule compartment wall material, or in the case of an end compartment which is closed by a closure, by the nature of the closure material. For example the wall of different, e.g. adjacent, compartments may be made of polymers which are different or which otherwise differ in their dissolution or disintegration characteristics so as to endow different compartments with different drug release characteristics. Similarly for example the polymer matrix material of different, e.g. adjacent, solid sub-units may be made of polymers which are different or which otherwise differ in their dissolution or disintegration characteristics so as to endow different solid sub-units with different drug release characteristics.

For example the matrix, wall or closure material may be a polymer which dissolves or disperses at stomach pH to release the drug substance in the stomach. Alternatively the wall material of different compartments may differ so that different compartments have different release characteristics.

For example a linker or closure sub-unit or a capsule compartment may have respectively a matrix or a wall or a closure comprising an enteric polymer which dissolves or disperses at the pH of the small or large intestine to release the drug substance in the intestine. Suitable such polymers have been described above, for example, with reference to U.S. Pat. No. 5,705,189.

Additionally or alternatively the wall material may differ in thickness between compartments so that thicker walled compartments disrupt more slowly than thinner walled compartments.

Additionally or alternatively the compartment walls or the closure may have areas or points of weakness which preferentially dissolve and may thereby determine the time of onset and/or rate of release of the drug substance content. For example such points of weakness may comprise holes, e.g. small holes, e.g. laser-drilled holes in the compartment wall or the closure, these holes being closed and/or covered with a film of a polymer material that dissolves at a pre-determined point in the digestive tract, for example an enteric polymer material. For example such points of weakness may comprise thinned parts in a capsule compartment wall formed during the molding operation in which the capsule compartment is formed.

The sub-units may additionally or alternatively have surface or other constructional features that modify their drug release characteristics. For example solid sub-units may be provided with internal cavities or channels to create a large surface area. For example solid sub-units may be in the form of hollow cylinders, donuts, or toroids, which shapes are known to tend towards first-order dissolution or erosion in liquid media and correspondingly to tend toward first-order release of drug content dispersed therein.

“Pharmaceutically acceptable agents” includes, but is not limited to, drugs, proteins, peptides, nucleic acids, nutritional agents, as described herein. This term includes therapeutic active agents, bioactive agents, active agents, therapeutic agents, therapeutic proteins, diagnostic agents, or drug(s) as defined herein, and follows the guidelines from the European Union Guide to Good Manufacturing Practice. Such substances are intended to furnish pharmacological activity or other direct effect in the diagnosis, cure, mitigation, treatment, or prevention of a disease or to affect the structure and function of the body. The substance may also include a diagnostic agent, such as an imaging agent and/or a radioactive labeled compound. Their use may be in a mammal, or may be in a human. The pharmacological activity may be prophylactic, or for treatment of a disease state. The agents herein include small molecule therapeutics, as well as peptides and proteins. The pharmaceutical compositions described herein may optionally comprise one or more pharmaceutically acceptable active agent, bioactive agent, active agent, therapeutic agent, therapeutic protein, diagnostic agent, or drug(s) or ingredients distributed within.

As used herein the term's “active agent”, “drug moiety” or “drug” are all used interchangeably.

The terms “mold” and “mould” are used interchangeably herein.

Water solubility of an active agent is defined by the United States Pharmacoepia. Therefore, active agents which meet the criteria of very soluble, freely soluble, soluble and sparingly soluble as defined therein are encompassed this invention.

Suitable drug substances can be selected from a variety of known classes of drugs including, but not limited to, analgesics, anti-inflammatory agents, anthelmintics, anti-arrhythmic agents, antibiotics (including penicillin's), anticoagulants, antidepressants, antidiabetic agents, antiepileptics, antihistamines, antihypertensive agents, antimuscarinic agents, antimycobactefial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, corticosteroids, cough suppressants (expectorants and mucolytics), diagnostic agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radiopharmaceuticals, sex hormones (including steroids), anti-allergic agents, stimulants and anorexics, sympathomimetics, thyroid agents, PDE IV inhibitors, NK3 inhibitors, CSBP/RK/p38 inhibitors, antipsychotics, vasodilators and xanthines.

Preferred drug substances include those intended for oral administration and intravenous administration. A description of these classes of drugs and a listing of species within each class can be found in Martindale, The Extra Pharmacopoeia, Twenty-ninth Edition, The Pharmaceutical Press, London, 1989, the disclosure of which is hereby incorporated herein by reference in its entirety. The drug substances are commercially available and/or can be prepared by techniques known in the art.

The polymeric blends can be preferably selected from known pharmaceutical polymers. The physico-chemical characteristics of these polymers, as well as the thickness of the ultimate injection molded component, will dictate the design of the dosage form, such as rapid dissolve, immediate release, delayed release, modified release such as sustained release, controlled release, or pulsatile release, etc.

The polymer blends are made by well-known methods for producing hot melt extrusions in which the selected ingredients are fed into a feed hopper of an extrusion machine. Suitable well known equipment is readily available for producing a hot melt extrusion of the blends herein.

The present invention is directed to a formulation that once moulded into a suitable capsule shell and/or linker does not require a film coating for acidic protection. Suitably, the desired subunit, e.g. a capsule shell, linker, endcap, etc. can be assembled by mechanical fit, and can readily be combined with another enteric shells or with another shell formulations to give a range of release profiles in a single unit.

Suitable formulation are chosen on the basis of acceptable performance in a number of factors, such as but not limited to:
1. Suitability for extrusion;
2. Ability to be injection moulded in a variety of subunits or components;
3. Physically stable (no warping, shrinking, cracking, etc.);
4. Chemically stable with respect to the polymers present;
5. Able to assembled manually/automatically (clipped) to a linker or solid matrix subunit;
6. Survive at least 2 hours in acid media with no release taking place; and
7. Dissolve/release at a pH above 6 in less than 45 minutes

Another aspect of this invention therefore, is a multicomponent dosage form which contains a capsule shell produced in accordance with the formulations described herein, and a suitable linker formulation, which can be easily assembled, such as by mechanical force, e.g. clipping, or by welding if desired, and which dosage form does not require any additional manipulation, such as external coating to provide an enteric release profile. Such multicomponent dosage form could be further extended to include an immediate release or a second pulsatile capsule or linker component as desired.

Therefore, one aspect of the invention is a multicomponent dosage form comprising a plurality of sub-units, and wherein each sub-unit being selected from

(a) at least one shell including a first wall portion at least partially defining an interior space configured to hold a drug substance, the first wall portion being configured to dissolve within a gastrointestinal environment; and

(b) at least one linker including a second wall portion having a substantially cylindrical outer surface, the second wall portion configured to dissolve within a gastrointestinal environment;

and wherein the drug substance containing capsule has a shell wall comprising hydroxypropylmethylcellulose acetate succinate (HPMC-AS) present in an amount of about 10 to 70% w/w, a plasticizer present in an amount of about 1% to about 20% w/w, a lubricant present in an amount of about 2% to about 15% w/w, and a swellable solid present in an amount of about 10 to about 60% w/w, and containing a drug substance; which, at least prior to administration to a patient, is mechanically welded or mechanically joined into an assembled dosage form.

A suitable linker or connecting sub-unit for use in the multicomponent dosage form is composed of ethylcellulose, stearyl alcohol, glycerol, and BHT (butylated hydroxytoluene).

Another suitable linker or connecting sub-unit for use in the multicomponent dosage form is composed of Eudragit RL100, HPC, and Stearyl alcohol.

Suitably, at least one of the capsule components in the multicomponent dosage form is of substantially sustained release, and any second capsule shell in the multicomponent dosage form may or may not be a formulation of the present invention.

Suitably, at least one of the multicomponent dosage form according to any of the preceding claims which further comprises a second drug substance-containing capsule compartment which is a substantially immediate release.

### EXAMPLES

The invention will now be described by reference to the following examples, which are merely illustrative and are not to be construed as a limitation of the scope of the present invention. All temperatures are given in degrees centigrade; all solvents are highest available purity unless otherwise indicated.

Table 1 provides a summary of formulations made and tested containing HPMC-AS.

TABLE 1

| | % w/w in formulation | | | |
|---|---|---|---|---|
| | HPMC | | Stearyl | Dissolution modifier |
| # | AS-LG | Triacetin | alcohol | (polymer) |
| 1 | 67.5 | 22.5 | 10 | 0 |
| 2 | 90 | 0 | 10 | 0 |
| 3 | 80 | 10 | 10 | 0 |
| 4 | 85 | 5 | 10 | 0 |
| 5 | 60 | 10 | 10 | 20 (Klucel ® EF) |
| 6 | 60 | 10 | 2.5 | 27.5 (Klucel ® EF) |
| 7 | 60 | 20 | 0 | 20 (Klucel ® EF) |
| 8 | 60 | 0 | 20 | 20 (Klucel ® EF) |
| 9 | 60 | 10 | 10 | 20 (Pharmacoat ® 603) |
| 10 | 40 | 2.5 | 10 | 47.5 (Klucel ® EF) |
| 11 | 55 | 10 | 2.5 | 32.5 (Klucel ® EF) |
| 12 | 40 | 17.5 | 2.5 | 40 (Klucel ® EF) |
| 13 | 70 | 6.25 | 6.25 | 17.5 (Klucel ® EF) |
| 14 | 70 | 10 | 10 | 10 (Klucel ® EF) |
| 15 | 62.5 | 13.75 | 4.38 | 19.38 (Klucel ® EF) |
| 16 | 40 | 10 | 6.25 | 43.75 (Klucel ® EF) |
| 17 | 55 | 13.75 | 6.25 | 25 (Klucel ® EF) |
| 18 | 70 | 13.75 | 2.5 | 13.75 (Klucel ® EF) |
| 19 | 55 | 6.25 | 10 | 28.75 (Klucel ® EF) |
| 20 | 70 | 10 | 10 | 10 (Klucel ® EF) |
| 21 | 62.5 | 6.25 | 8.13 | 23.13 (Klucel ® EF) |
| 22 | 55 | 10 | 2.5 | 32.5 (Klucel ® EF) |
| 23 | 55 | 2.5 | 10 | 32.5 (Klucel ® EF) |
| 24 | 40 | 10 | 10 | 40 (Klucel ® EF) |
| 25 | 70 | 17.5 | 2.5 | 10 (Klucel ® EF) |
| 26 | 70 | 6.25 | 6.25 | 17.5 (Klucel ® EF) |
| 27 | 40 | 10 | 10 | 40 (Klucel ® EF) |
| 28 | 47.5 | 10 | 4.38 | 38.13 (Klucel ® EF) |

Formulations #1, 2, 4 and 7 have been determined to be inoperative;
formulation #26 was also found to be inoperative and is an example of the ratio of components lying outside the desired 8:1 ratio.

Table 2 provides a summary of formulations made with HPMC AS-MG or HPMC AS-HG as the base polymer.

TABLE 2

| | % w/w in formulation | | | | |
|---|---|---|---|---|---|
| | HPMC AS | | | Stearyl | Dissolution modifier |
| # | (grade) | | Triacetin | alcohol | (polymer) |
| 29 | 90 | (MG) | 10 | 0 | 0 |
| 30 | 60 | (MG) | 10 | 10 | 20 (Klucel ® EF) |
| 31 | 60 | (HG) | 10 | 10 | 20 (Klucel ® EF) |

Formulation #29, in the above table, was determined to be inoperative.

Additional formulations of stearyl alcohol and HPMC-AS:triacetin have been formulated and have been shown to provide physical stability at 40/75° C. Using an HPMC-AS:triacetin ratio of 7:1, the following HPMC-AS (LG) shell formulations have completed 1 month on storage at 30/65° C. and 40/75° C., and have been shown to be physically stable with minimum colour change.

TABLE 3

| | |
|---|---|
| 1. | 70% HPMC-AS (LG)/13.75% HPC (such as EF)/6.25% SA/10% |
| | Triacetin |
| 2. | 60% HPMC-AS (LG)/25.18% HPC (such as EF)/6.25% SA/8.57% |
| | Triacetin |
| 3. | 50% HPMC-AS (LG)/36.61% HPC (such as EF)/6.25% SA/7.14% |
| | Triacetin |
| 4. | 40% HPMC-AS (LG)/48.04% HPC (such as EF)/6.25% SA/5.71% |
| | Triacetin |
| 5. | 30% HPMC-AS (LG)/59.46% HPC (such as EF)/6.25% SA/4.29% |
| | Triacetin |
| 6. | 20% HPMC-AS (LG)/70.89% HPC (such as EF)/6.25% SA/2.86% |
| | Triacetin |

Other embodiments of the invention include the additional formulations in Table 4 which have been extruded and injection moulded.

TABLE 4

| | |
|---|---|
| 1. | HPMC-AS (LG) 62.75%, 21% HPC (such as SSL)/6.25% SA/10% |
| | glycerol |
| 2. | HPMC-AS (LG) 62.75%, 24.5% HPC (such as EF)/6.5% SA/6.25% |
| | glycerol |
| 3. | HPMC-AS (LG) 62.75%, 24.5% HPC (such as SSL)/6.5% SA/6.5% |
| | TEC |
| 4. | HPMC-AS (LG) 62.75%, 24.5% HPC (such as EF)/6.5% SA/6.5% |
| | TEC |

The following additional tables, Tables 5 to 10 demonstrate representative compositions of the instant invention which may be suitably extruded and moulded into dosage form components as described herein. The formulations are all expressed in w/w % amounts. All formulations have been extruded and injection moulded into capsule shells using the general hot melt extrusion and injection moulding methods discussed below.

TABLE 5

| | | HPC | HPC | HPMCP | | | | |
|---|---|---|---|---|---|---|---|---|
| | HPMC | (such as | (such as | (such as HP | | | | Stearyl |
| # | AS-LG | Klucel EF | SSL) | 50/55S/55) | Glycerol | TEC | Triacetin | alcohol |
| 1 | 60 | 20 | | | | | 10 | 10 |
| 2 | 60 | 27.5 | | | | | 10 | 2.5 |
| 3 | 40 | 40 | | | | | 17.5 | 2.5 |
| 4 | 62.75 | 24.5 | | | | | 6.5 | 6.25 |
| 5 | 62.75 | 24.5 | | | 6.5 | | | 6.25 |
| 6 | 62.75 | 24.5 | | | | 6.5 | | 6.25 |
| 7 | 62.75 | | 24.5 | | | 6.5 | | 6.25 |
| 8 | 62.75 | | 24 | | 10 | | | 3.25 |
| 9 | 62.75 | 24 | | | 10 | | | 3.25 |
| 10 | 59.5 | 22 | | | | | 12 | 6.5 |
| 11 | 59 | | | 24.5 (50) | 10 | | | 6.5 |
| 12 | 59 | | | 24.5 (55S) | 10 | | | 6.5 |
| 13 | 59 | | | 24.5 (50) | | 10 | | 6.5 |
| 14 | 59 | | | 24.5 (50) | | | 10 | 6.5 |
| 15 | 59 | | | 19.5 (50) | | | 15 | 6.5 |
| 16 | 59 | | | 19.5 (50) | | 15 | | 6.5 |
| 17 | 41 | | | 40.5 (50) | | | 12 | 6.5 |
| 18 | 59 | | | 19.5 (55S) | | | 15 | 6.5 |
| 19 | 62.75 | 24.5 | | | 6.25 | | | 6.5 |
| 20 | 50 | | 15 | 15 (50) | 5 | 10 | | 5 |
| 21 | 61.4 | 18.9 | | | 13 | | | 6.7 |
| 22 | 70.1 | 10.2 | | | 13 | | | 6.7 |

TABLE 6

| | | | | HPMCP | | | | | HPMC |
|---|---|---|---|---|---|---|---|---|---|
| | | HPC | | (such | | | | | (such as |
| | HPMC | (such as | HPMCP | as HP | Glyc- | | Propylene | Stearyl | Pharma- |
| # | AS-LG | SSL) | (HP 50) | 55S/55) | erol | TEC | Glycol | alcohol | coat 603) |
| 1 | 61.4 | | 20.5 | | 4.3 | | 8.7 | 5.1 | |
| 2 | 60 | | 20 | | 5 | | 10 | 5 | |
| 3 | 60 | | 20 | | | 5 | 10 | 5 | |
| 4 | 59 | | 18.5 | | 7.5 | | 10 | 5 | |
| 5 | 57 | 3 | 20 | | 5 | | 10 | 5 | |
| 6 | 60 | | 20 | | | 5 | 10 | 5 | |
| 7 | 74 | | | | 5 | | 10 | 6 | 5 |
| 8 | 56 | | | 18 (55) | 5 | | 10 | 6 | 5 |
| 9 | 56 | | | 18 (55S) | 5 | | 10 | 6 | 5 |
| 10 | 56 | | 18 | | 5 | | 10 | 6 | 5 |
| 11 | 53 | 3 | 18 | | 5 | | 10 | 6 | 5 |
| 12 | 60 | | 20 | | | 5 | 10 | 5 | |
| 13 | 57 | 3 | 20 | | 5 | | 10 | 5 | |
| 14 | 50 | 7 | 20 | | 5 | | 10 | 5 | 3 |
| 15 | 45 | 20 | 15 | | 5 | | 10 | 5 | |
| 16 | 45 | 20 | 15 | | 10 | | 5 | 5 | |
| 17 | 50 | 20 | 15 | | 5 | | 5 | 5 | |
| 18 | 50 | 15 | 15 | | 5 | | 10 | 5 | |
| 19 | 59 | | 19.5 | | | 10 | 5 | 6.5 | |
| 20 | 56.2 | | 18.5 | | | 9.5 | 4.8 | 6.2 | 4.8 |
| 21 | 58.5 | 3 | 18.5 | | 5 | | 10 | 5 | |
| 22 | 56 | | 18 | | 5 | | 10 | 6 | 5 |

TABLE 7

| | | HPMCP | | | | | | | |
|---|---|---|---|---|---|---|---|---|---|
| | HPMC | (such as | | | Propylene | | Stearyl | | Yellow |
| # | AS-LG | HP 50) | TEC | Triacetin | Glycol | ATBC | alcohol | TiO2 | FeO |
| 1 | 58.5 | 20 | | 10 | 5 | | 6.5 | | |
| 2 | 58 | 19.5 | | 11 | 4 | | 6.5 | .75 | .25 |
| 3 | 58 | 19.5 | 4 | 11 | | | 6.5 | .75 | .25 |
| 4 | 60 | 19 | | 15 | | | 5 | .75 | .25 |
| 5 | 58 | 19.5 | | 15 | | | 6.5 | .75 | .25 |
| 6 | 61.75 | 20.75 | | | 10 | | 6.5 | .75 | .25 |
| 7 | 39 | 39 | | 15 | | | 6 | .75 | .25 |
| 8 | 61.75 | 20.75 | | | | 10 | 6.5 | .75 | .25 |
| 9 | 58 | 19.5 | | | | 15 | 6.5 | .75 | .25 |
| 10 | 58 | 19.5 | | 15 | | | 6.5 | .75 | .25 |

TABLE 8

| | | | | | | | | | | Surfac- |
|---|---|---|---|---|---|---|---|---|---|---|
| | | HPC | HPC | | | | | | | tant |
| | | (such as | (such | | | | | | | (such as |
| | HPMC | Klucel | as | HP | Glyc- | Peg | Tartaric | Stearyl | | Tween |
| # | AS-LG | EF) | SSL) | 50 | erol | 400 | acid | alcohol | CaCO3 | 80) |
| 1 | 62.75 | 19.5 | | | 6.5 | | 5 | 6.25 | | |
| 2 | 61.75 | | 20.75 | | | 10 | | 6.5 | | 1 |
| 3 | 60.75 | | | 20.75 | | 10 | | 6.5 | 2 | |
| 4 | 59 | | | 19.5 | | 15 | | 6.5 | | |
| 5 | 62.75 | | 24.5 | | | 6.25 | | 6.5 | | |

TABLE 9

| | | HPC | | | | | | | |
|---|---|---|---|---|---|---|---|---|---|
| | HPMC | (such as | HPC (such | | | Stearyl | | | |
| # | AS-LG | Klucel EF) | as SSL) | Glycerol | Triacetin | alcohol | SDS | TiO2 | BHT |
| 1 | 62.75 | 21.75 | | | 8 | 6.5 | | 1 | |
| 2 | 62.75 | 21.75 | | 8 | | 6.5 | | 1 | |
| 3 | 62.75 | | 21.75 | 8 | | 6.5 | | 1 | |
| 4 | 62.75 | 20 | | 10 | | 6.25 | 1 | | |
| 5 | 62.75 | 23 | | 10 | | 3.25 | 1 | | |
| 6 | 62.75 | | 20 | 10 | | 6.25 | 1 | | |
| 7 | 62.75 | | 23 | 10 | | 3.25 | 1 | | |
| 8 | 59 | 20 | | | 10 | 20 | | | 1 |

TABLE 10

| | | | | | Stearyl | Amberlite | | Yellow |
|---|---|---|---|---|---|---|---|---|
| # | HPMC AS-LG | HP 50 | Triacetin | Miglyol | alcohol | IRP88 | TiO2 | FeO |
| 1 | 59 | 17 | 15 | | 5 | 3 | .75 | .25 |
| 2 | 61.75 | 20.75 | | 10 | 6.5 | | .75 | .25 |
| 3 | 59 | 24.5 | | 10 | 6.5 | | | |

### Hot-Melt Extrusion

Prior to hot melt extrusion, the powder excipients of the above noted formulations, (HPMC AS, stearyl alcohol and dissolution modifying polymer) were blended via a bin blender. The extrusion was generically performed on a Prism 16 mm co-rotating twin-screw extruder with a temperature profile range from die to feed throat of 120-120-115-110-90-20° C. and screw speed of 200 rpm. It is possible that the temperature range for the above examples may have varied by 10° C.+/−. The extruder was fed by a gravimetric powder feeder and the triacetin which is a liquid was added via a Gilston Minipuls 2 peristaltic pump, total combined feed rate was set to equal approximately 1.0 kg/hr. The formulations were extruded through a 3 mm die to produce a strand that was then air cooled and then palletized.

### Injection Moulding

The pellets produced from the hot-melt extrusion process above were injection moulded using an MCP 12/90 HSP mini moulder into prototype 9.0 mm diameter×6.9 mm height capsule shell, with a wall thickness of 0.5 mm; or a 7.7 mm diameter×9 mm height capsule shell. Typically the screw, plunger and barrel temperature was set to 120-140° C. and a probe temperature to 170 to about 190° C. as the upper temperature.

It has been found that dissolution testing, either for enteric protection or for release alone is not a good predictor of a formulations acceptance in many cases. A number of formulations provide similar release profiles and additional investigations into stability or tensile strength may be necessary.

Enteric shells may be tested using at least three known methods of dissolution tests, a USP II paddle method to simulate the standard USP enteric protection test using a pH switch method to coincide with testing typically performed for enteric dosage forms; a USP III test, which may be more bio-relevant, using a set agitation rate of 10 dips per minute (DPM); and a 2 hour enteric challenge, as this test is more relevant to in vivo performance (as no sinkers are used and the dosage form can float and sink).

The USP III is also used to determine the maximum amount of time a formulation could withstand an acidic environment before releasing. This test does make an assumption that not all units would exit the stomach in a rapid manner and could be retained for extended periods of time (in the stomach) before getting to a high pH intestinal region. Therefore this test is performed by holding the unit in the low pH phase for at least 6 hours and checking for release.

### USP II Methodology

The injection moulded shells are dosed with paracetamol, as a marker drug, and sealed by clipping to an 8.35 mm diameter, 3.80 mm height injection moulded linker unit. Dissolution analysis is performed via the USP2 paddle method at 100 revolutions per minute, with 2 hours in pH 1.6 0.1N hydrochloric acid followed by 2 hours in pH 6.8 phosphate buffer with 0.06% sodium dodecyl sulphate, and units were placed in the vessels in Japanese cage sinkers.

### USP III Methodology

The injection moulded shells are dosed with metformin, as a marker drug, and sealed by clipping to an 8.35 mm diameter, 3.80 mm height injection moulded linker unit. Dissolution analysis is performed via USP3 method at 10 dips per minute, with 2 hours in pH 1.2 simulated gastric fluid followed by 6 hours in pH 6.8 simulated intestinal fluid with the units placed in the baskets without sinkers.

The methods above use either pH 1.2 SGF or pH 1.6 0.1N HCl as the acidic phase, and either pH 6.8 SIF or pH 6.8 phosphate buffer. These mediums are essentially interchangeable so long as the pH is kept low in the acidic phase (ideally <2) and the high pH phase is above the pH dissolution threshold for the enteric polymers present which in most cases is above pH 5.5.

### USP II/III of Final IR/ER Formulations

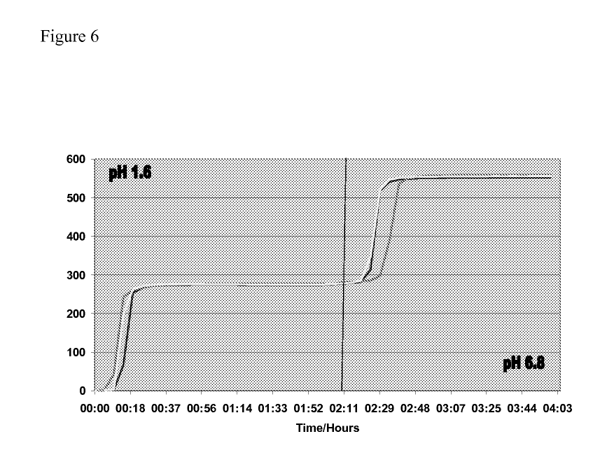

To confirm completed units with different formulated shells can provide variable release profiles from a single dosage form, shells are tested with an immediate release shells of the same dimensions. FIG. 6 demonstrates a typical USP II plot showing complete units comprising of IR and enteric shells.

Injection moulded shells which were dosed with Metformin, used as a marker drug, were sealed by clipping to an 8.35 mm diameter, 3.80 mm height injection molded linker unit unless otherwise indicated. Dissolution analysis was performed via USP3 method at 10 dips per minute, with 2 hours in pH 1.2 simulated gastric fluid followed by 6 hours in pH 6.8 simulated intestinal fluid with the units placed in the baskets without sinkers.

The following linker formulations were used:

Linker composition:
Ethylcellulose (N22 grade, Aqualon) 84% (all w/w)
Stearyl alcohol 10%
Glycerol 5%
BHT (butylated hydroxytoluene) 1%

Extrusion of this linker component was performed using a 16 mm twin-screw extruder at temperatures ranging between 120-130 degrees ° C. and samples were moulded to form linker shaped components at temperatures between 160-180° C.

RL Linker composition:
Eudragit RL100 25.00% w/w
HPC such as Klucel EF 63.00
Stearyl alcohol 12.00

Process Conditions

Extrusion/Injection moulding: Extrusion—1.2 kg/hr die temp. 110° C., 200 rpm screw, torque 35%, die pressure 1 bar; Injection Moulding—satisfactory 0.5 mm wall section shells, 180 C probe temp.

### Alternative Linker Formulations:

Using a higher molecular weight grade of HPMC-AS HG (dissolves at 6.5-7.0), in combination with two levels of the plasticizer triacetin and the lubricant stearyl alcohol, two formulations have been made and tested.

Content in Formulation (% w/w)

| | A | B |
|---|---|---|
| HMPC AS-HG | 90 | 85 |
| Triacetin | 5 | 10 |
| Stearyl Alcohol | 5 | 5 |

Similar formulations using HPMC-AS LG (pH 5.5) and MG (pH 5-5.6) with 5% w/w levels of triacetin have also been made.

Linkers were also produced from the HPMC-AS HG polymer using two alternative plasticizers, glycerol and triethyl citrate. In both cases, the alternative plasticizer was added at a 5% w/w level. Stearyl alcohol was present in both cases at the 5% w/w level. It was noted that the maximal extruder torque for the glycerol formulation was exceeded and the glycerol level had to be increased to 10% w/w to match the torque produced by the triacetin plasticized formulation, suggesting that glycerol was a less effective plasticizer than triacetin for this composition. In contrast, the triethyl citrate formulation ran at a similar torque to the comparative triacetin formulation.

The extrusion of linker blends was performed on a Prism 16 mm co-rotating twin-screw extruder with a temperature profile from die to feed throat of 120-120-115-110-90-20° C. and screw speed of 200 rpm. The extruder was fed by a gravimetric powder feeder and the triacetin which is a liquid was added via a Gilston Minipuls 2 peristaltic pump, total combined feed rate was set to equal approximately 1.0 kg/hr. The formulations were extruded through a 3 mm die to produce a strand that was then air cooled and then palletized.

### Results

Generally the formulations could be extruded and formed strands that were suitable for pelletisation. The level of plasticizers in the formulation assists in determining the overall flexibility of the shells. It is recognized that some of the formulations described while mouldable, may produce parts which have characteristics that made them unsuitable for commercialization, e.g. they produce parts that are too brittle to clip, are prone to frequent cracking, and/or have excessive stretching.

A lubricant is deemed is deemed necessary for formulations herein as removal of the lubricant, e.g. stearyl alcohol, produces parts that stick in the mould cavities. Stearyl alcohol was removed completely from Formulation 5, Table 1 to assess whether the triacetin could be used alone to lower the melt viscosity for injection moulding and extrusion. Complete parts could not be formed from this formulation, suggesting an inadequate amount of plasticizer. In formulation 11, Table 1, the level of plasticizer was increased, and this produced shells that stuck in the mould cavities. The removed shells were complete and very flexible but were deemed inelastic and did not return to their shape upon deformation suggesting the shells were over-plasticized.

A suitable plasticizer level is required to produce parts which have flexibility, such as for attachment to a linker, which produce stable components, and which are dimensionally correct. This is exemplified herein with observations of formulations 3 and 4 in Table 1.

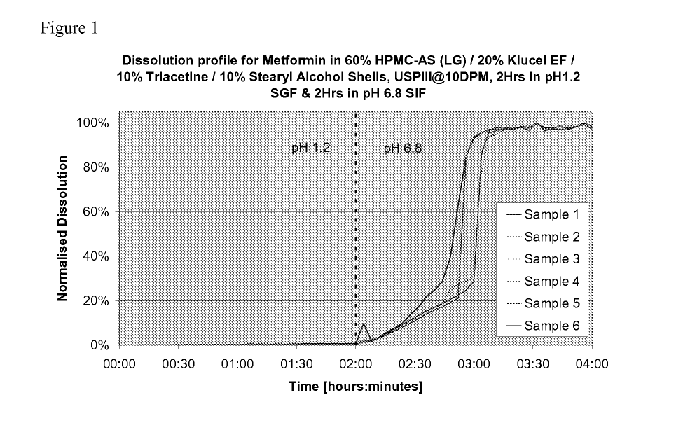

Dissolution analysis of the unit from Formulations 3 and 4, Table 1 showed gastric resistance for 2 hours and release in simulated intestinal fluid. Formulation 4 produced variable dissolution profiles; which variability was reduced in Formulation 3. The addition of HPC (Formulation 6, Table 1) to the formulation allowed the units to partially hydrate in the gastric fluid. This reduces the time to release in the intestinal fluid while still providing gastric resistance. Dissolution analysis with 8 hours in gastric fluid showed that after extended gastric residence the units although hydrated, continue to maintain gastric resistance. This formulation was repeated with the HPMC-AS grade substituted for MG and HG. The different grades demonstrated extend the time to release in the intestinal fluid. FIG. 1 demonstrates a dissolution profile of a 60% HPMC-AS (LG)/20% Klucel EF/10% Triacetin/10% stearyl alcohol shell (a 60:20:10:10 formulation) in simulated gastric fluid.

These formulations survive exposure to SGF fluids for about 2 hours, and then release within 1 hour of exposure to SIF. For the most part, linkers used with capsule shell components of this invention are desired to be insoluble under gastric conditions, thus providing a dosage form which releases as a pulsatile release dosage form in the small intestines.

Additional USP 3 release times for representative formulations of the invention are shown below. These release times, are a ‘typical’ release for each formulation. Only USP 3 data is shown at the same run conditions, and using the same size/wall section shells and the same linkers, in this instance a RL100 linker composition, for all units to provide comparison of the formulations.

The results described herein do not provide a full and conclusive set for all formulations made, and are only a representative release time for comparative purposes. The table below is one such representative sample:

| | Enteric Protection | USP3 Release | |
|---|---|---|---|
| Formulation | (>2 hrs) | times at pH 6.8 | |
| HPMC-AS/HP-50/Stearyl | Yes | 24-36 | minutes |
| alcohol/HPC-SSL/Glycerol/ | | | |
| Propylene Glycol | | | |
| (58.5/18.5/5/3/5/10% w/w) | | | |
| HPMC-AS/HP-50/Stearyl | Yes | 36-72 | minutes |
| alcohol/Pharmacoat 603/ | | | |
| Glycerol/Propylene Glycol | | | |
| (56/18/6/5/5/10% w/w) | | | |
| HPMC-AS/Stearyl alcohol/ | Yes | 44-80 | minutes |
| Pharmacoat 603/Glycerol/ | | | |
| Propylene Glycol | | | |
| (74/6/5/5/10% w/w) | | | |
| HPMC-AS/HP-55/Stearyl | Yes | 48-92 | minutes |
| alcohol/Pharmacoat 603/ | | | |
| Glycerol/Propylene Glycol | | | |
| (56/18/6/5/5/10% w/w) | | | |
| HPMC-AS/HP-50/PEG | Yes | 40-64 | minutes |
| 400/Stearyl alcohol | | | |
| (59/19.5/15/6.5% w/w) | | | |
| HPMC-AS/HP-50/Stearyl | Yes | 44-64 | minutes |
| alcohol/Triethyl | | | |
| Citrate/Propylene | | | |
| Glycol/Pharmacoat 603 | | | |
| (56.2/18.5/6.2/9.5/4.8/4.8% | | | |
| w/w) | | | |
| HPMC-AS/HP-50/Triacetin/ | Yes | 48-64 | minutes |
| Stearyl alcohol | | | |
| (59/19.5/15/6.5% w/w) | | | |
| HPMC-AS/HPC-SSL/Stearyl | No/Variable | Not applicable | |
| alcohol/SDS/Glycerol | | (already | |
| (62.75/20/6.25/1/10% w/w) | | released) | |
| HPMC-AS/Klucel EF/Stearyl | Yes | 44-68 | minutes |
| alcohol/Glycerol | | | |
| (62.75/24.5/6.5/6.25% w/w) | | | |
| (Run with Ethyl Cellulose based | | | |
| linkers) | | | |
| HPMC-AS/Klucel EF/Stearyl | Yes | 60-120+ | minutes |

alcohol/TiO2/Triacetin
(62.75/21.75/6.5/1/8% w/w)

A number of different linker variants (e.g. RL100, ethylcellulose, and HPMC-AS) have been tried with the formulations of the present invention. The RL100 linkers have demonstrated a tendency to swell and hydrate over longer periods of time. While this may not be an issue with formulations suitable for immediate release it is possible that this might be an issue with the enteric units retained in the stomach for long periods of time as release could occur through the linker rather than through the shell dissolution.

Previously moulded HPMC-AS linkers have generally been found to be smaller than RL100 linkers, and do not swell as well resulting in linkers which fall out of the shells before they have time to fully dissolve, often ending in enteric failures. HPMC-AS and ethylcellulose linkers however, can be used with formulations containing HPC such as those with Klucel EF at around 20% w/w as these formulations have been found to hydrate and swell in acidic media, and therefore hold the linker into the shell to prevent premature release. This can turn a formulation with a previously long lag in release to a much quicker and more reproducible release.

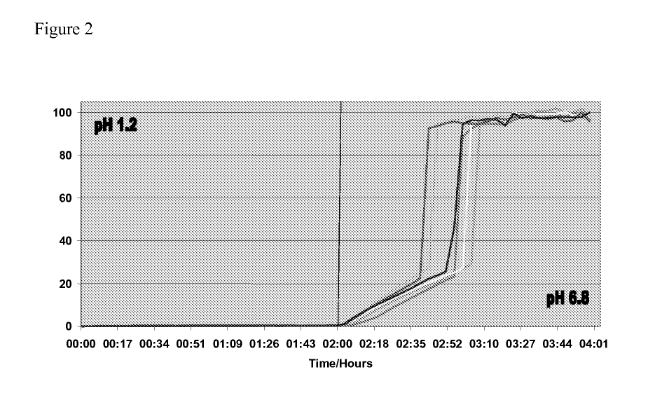

The dissolution profile of enteric shells containing Klucel EF, which is known to swell, will slow down the release rate when used with RL100 linkers. In contrast, using ethyl cellulose linkers the release is much faster and more consistent, going from 60-120 minutes with an RL100 linker to 44-68 minutes with an ethyl cellulose linker as can be seen in FIG. 2 herein.

The above description fully discloses the invention including preferred embodiments thereof. Modifications and improvements of the embodiments specifically disclosed herein are within the scope of the following claims. Without further elaboration, it is believed that one skilled in the area can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A dosage form comprising at least one of:
(a) a shell including a first wall portion at least partially defining an interior space configured to hold a drug substance, the first wall portion being configured to dissolve within a gastrointestinal environment; or
(b) a linker including a second wall portion having a substantially cylindrical outer surface, the second wall portion configured to dissolve within a gastrointestinal environment;
wherein a respective one of the first or second wall portions are made from an extruded pharmaceutical composition comprising hydroxypropylmethylcellulose acetate succinate (HPMC-AS) present in an amount of about 20 to about 70% w/w; at least one plasticizer present in an amount of about 1% to about 20% w/w; a lubricant present in an amount of about 2% to about 10% w/w; and at least one dissolution modifying excipient selected from the group consisting of a disintegrant present it is in an amount of about 2% to about 20% w/w, a swellable solid present it is in an amount of about 10 to about 60% w/w, and a wicking agent present it is in an amount of about 2.5 to about 15% w/w, and a combination or mixture thereof.

2. The dosage form according to claim 1 wherein the HPMC-AS is present in an amount of about 55 to about 65% w/w.

3. The dosage form according to claim 1 wherein the lubricant is stearyl alcohol, glycerol monostearate (GMS), talc, magnesium stearate, silicon dioxide, amorphous silicic acid, or fumed silica; and combinations or mixtures thereof.

4. The dosage form according to claim 3 wherein the lubricant is stearyl alcohol.

5. The dosage form according to claim 4 wherein the stearyl alcohol is present from about 4 to about 10% w/w.

6. The dosage form according to claim 1 wherein the at least one dissolution modifying excipient is a swellable solid.

7. The dosage form according to claim 6 wherein the swellable solid is at least one of hydroxypropyl cellulose, hydroxypropylmethyl cellulose, or hydroxypropylmethyl cellulose phthalate, or a combination or mixture thereof.

8. The dosage form according to claim 7 wherein the swellable solid is a combination of hydroxypropyl cellulose and hydroxypropylmethyl cellulose.

9. The dosage form according to claim 7 wherein the swellable solid is a combination of hydroxypropyl cellulose and hydroxypropylmethyl cellulose phthalate.

10. The dosage form according to claim 6 wherein the swellable solid is a blend of hydroxypropyl cellulose polymers each having differing molecular weights.

11. The dosage form according to claim 10 wherein the blend of hydroxypropyl cellulose polymers are present in a total amount of about 20% to about 50% w/w.

12. The dosage form according to claim 1 wherein the at least one dissolution modifying agent is a wicking agent which is a low molecular weight solute or a sugar selected from xylitol, mannitol, lactose, starch, or sodium chloride, or combinations or mixtures thereof.

13. The dosage form according to claim 1 wherein the at least one dissolution modifying excipient is a disintegrant.

14. The dosage form according to claim 13 wherein the disintegrant is sodium starch glycollate, croscarmellose sodium, crospovidone (cross-linked polyvinyl pyrrolidone), copovidone, polyvinyl pyrrolidone, or a combination or mixture thereof.

15. The dosage form according to claim 14 wherein the at least one dissolution modifying excipient is a disintegrant present in an amount of about 5 to about 10% w/w.

16. The dosage form according to claim 1 wherein the plasticizer is selected from the group consisting of triacetin, triethyl citrate (TEC), tributyl citrate, acetyl triethyl citrate (ATEC), acetyl tributyl citrate (ATBC), dibutyl phthalate, dibutyl sebacate (DBS), diethyl phthalate, vinyl pyrrolidone glycol triacetate, polyethylene glycol, glycerol, polyoxyethylene sorbitan monolaurate, propylene glycol, and castor oil, or a combination or mixture thereof.

17. The dosage form according to claim 16 wherein the plasticizer is triacetin.

18. The dosage form according to claim 17 wherein the triacetin is present in an in a ratio with HPMC-AS of about 1:4 to 1:7.

19. The dosage form according to claim 16 wherein the plasticizer is triethyl citrate or glycerol.

20. The dosage form according to claim 16 wherein the plasticizer is a mixture of glycerol and propylene glycol.

21. The dosage form according to claim 16 wherein the plasticizer is a mixture of triethyl citrate and propylene glycol.

22. The dosage form according to claim 1 wherein the plasticizer is a mixture of two or more plasticizers present in an amount of about 10% w/w to about 20% w/w.

23. The dosage form according to claim 1 which further comprises a surfactant present in an amount of 1 to about 10%, and/or a processing agent present in an amount of about 1 to about 10% w/w.

24. The dosage form according to claim 1 wherein the lubricant is stearyl alcohol, the dissolution modifying excipient is HPC or a blend of differing molecular weights of HPC, and the plasticizer is TEC or triacetin.

25. The dosage form according to claim 1 wherein the grade of HPMC-AS is HPMC-AS LG.

26. The dosage form according to claim 1 wherein HPMC-AS is present in an amount of about 50 to about 65% w/w, the dissolution modifying excipient is HPMC phthalate present in an amount from about 10 to about 50% w/w, the lubricant is stearyl alcohol present in an amount of about 4 to about 10% w/w, and at least one plasticizer present in an amount of about 10 to about 20% w/w.

27. The dosage form according to claim 26 wherein the plasticizer is glycerol or propylene glycol, or a combination or mixture thereof.

28. The dosage form according to claim 26 wherein the plasticizer is TEC or propylene glycol, or a combination or mixture thereof.

29. The dosage form according to claim 1 wherein the at least one dissolution modifying excipient is a swellable solid which is HPC and a second swellable solid which is HPMC, present in the formulation in an amount of about 2 to about 10% w/w.

30. The dosage form according to claim 1 wherein the pharmaceutical composition comprises HPMC-AS, hypromellose phthalate, hydroxypropylcellulose, propylene glycol, glycerol, and stearyl alcohol.

31. The dosage form according to claim 1 wherein the HPMC-AS is LG grade.

32. The dosage form according to claim 1 wherein the stearyl alcohol is present in an amount of from about 3.75 to about 6.25% w/w.

33. The dosage form according to claim 30 wherein the HPMC-AS, hypromellose phthalate, hydroxypropylcellulose, propylene glycol, glycerol, and stearyl alcohol are present in the formulation as 58.5/18.5/3/10/5/5% w/w.

34. The dosage form according to claim 1 wherein the pharmaceutical composition comprises:
HPMC-AS/hypromellose phthalate/Stearyl alcohol/HPC-SSL/Glycerol/Propylene Glycol (58.5/18.5/5/3/5/10% w/w); or
HPMC-AS/Stearyl alcohol/hypromellose/Glycerol/Propylene Glycol (74/6/5/5/10% w/w); or
HPMC-AS/hypromellose phthalate/Stearyl alcohol/hypromellose/Glycerol/Propylene Glycol (56/18/6/5/5/10% w/w); or
HPMC-AS/hypromellose phthalate/PEG 400/Stearyl alcohol (59/19.5/15/6.5% w/w); or
HPMC-AS/hypromellose phthalate/Stearyl alcohol/Triethyl Citrate/Propylene Glycol/hypromellose (56.2/18.5/6.2/9.5/4.8/4.8% w/w); or
HPMC-AS/hypromellose phthalate/Triacetin/Stearyl alcohol (59/19.5/15/6.5% w/w); or
HPMC-AS/hydroxypropylcellulose/Stearyl alcohol/SDS/Glycerol (62.75/20/6.25/1/10% w/w); or
HPMC-AS/hydroxypropylcellulose/Stearyl alcohol/Glycerol (62.75/24.5/6.5/6.25% w/w); or
HPMC-AS/hydroxypropylcellulose/Stearyl alcohol/TiO2/Triacetin (62.75/21.75/6.5/1/8% w/w).

35. The dosage form according to claim 1 wherein HPMC-AS is present in an amount of about 50 to about 65% w/w.

36. The dosage form according to claim 1 wherein HPMC-AS is present in an amount of about 40 to about 70% w/w.

37. A dosage form comprising at least one of:
(a) a shell including a first wall portion at least partially defining an interior space configured to hold a drug substance, the first wall portion being configured to dissolve within a gastrointestinal environment; or
(b) a linker including a second wall portion having a substantially cylindrical outer surface, the second wall portion configured to dissolve within a gastrointestinal environment;
wherein a respective one of the first or second wall portions are made from an extruded pharmaceutical composition comprising hydroxypropylmethylcellulose acetate succinate (HPMC-AS) present in an amount of about 40 to 70% w/w, stearyl alcohol present in an amount of about 5 to about 10% w/w; a hydroxypropylcellulose derivative present in an amount of about 10 to about 50% w/w; and at least one plasticizer present in an amount of about 1 to about 30% w/w.

38. The dosage form according to claim 37 wherein the hydroxypropyl cellulose has a molecular weight of <130,000.

39. The dosage form according to claim 37 wherein the plasticizer is triacetin.

40. The dosage form according to claim 37 wherein the HPMC-AS is LG grade.

41. The dosage form according to claim 37 which is:

| | % w/w in formulation | | | |
|---|---|---|---|---|
| | | | Stearyl | |
| Example # | HPMC AS | Triacetin | alcohol | HPC |
| 1 | 67.5 | 22.5 | 10 | 0 |
| 2 | 90 | 10 | 0 | 0 |
| 3 | 80 | 10 | 10 | 0 |
| 4 | 85 | 5 | 10 | 0 |
| 5 | 90 | 10 | 0 | 0 |
| 6 | 60 | 10 | 10 | 20 |
| 7 | 60 | 10 | 2.5 | 27.5 |

42. A multicomponent dosage form comprising a plurality of sub-units, and wherein each sub-unit being selected from
(a) at least one shell including a first wall portion at least partially defining an interior space configured to hold a drug substance, the first wall portion being configured to dissolve within a gastrointestinal environment; and
(b) at least one linker including a second wall portion having a substantially cylindrical outer surface, the second wall portion configured to dissolve within a gastrointestinal environment;
and wherein the drug substance containing capsule has a shell wall comprising hydroxypropylmethylcellulose acetate succinate (HPMC-AS) present in an amount of about 20 to 70% w/w, at least one plasticizer present in an amount of about 1% to about 20% w/w, a lubricant present in an amount of about 2% to about 15% w/w, and a swellable solid present in an amount of about 10 to about 60% w/w, and containing a drug substance;
which, at least prior to administration to a patient, is mechanically welded or mechanically joined into an assembled dosage form.

43. The multicomponent dosage form according to claim 42 wherein the at least one linker is composed of ethylcellulose, stearyl alcohol, glycerol, and BHT (butylated hydroxytoluene).

44. The multicomponent dosage form according to claim 42 wherein the at least one linker is composed of Eudragit RL100, hydroxypropylcellulose and stearyl alcohol.

45. The multicomponent dosage form according to claim 42, in which the at least one drug substance-containing capsule compartment has a wall with a thickness in the range of about 0.1-0.8 mm.

46. The multicomponent dosage form according to claim 42, in which the at least one drug substance-containing capsule compartment is a substantially sustained release.

47. The multicomponent dosage form according to claim 42 which further comprises a second drug substance-containing capsule compartment which is a substantially immediate release.
